(19) United States
(12) Patent Application Publication
(10) Pub. No.: US 2006/0206089 A1
(43) Pub. Date: Sep. 14, 2006

(54) Absorbent article for incontinent women

(76) Inventor: Petriva W. Coley, Loxley, AL (US)

Correspondence Address: LITMAN LAW OFFICES, LTD, PO BOX 15035, CRYSTAL CITY STATION, ARLINGTON, VA, 22215, US

(21) Appl. No.: 11/074,698

(22) Filed: Mar. 9, 2005

(51) Int. Cl. A61F 13/20

(52) U.S. Cl. 604/385.17

(57) ABSTRACT

The absorbent article for incontinent women is a cylindrical roll of absorbent material that is disposed between the labia majora just under the urethra. The article absorbs urine accidentally discharged by the body. The article may be disposed in a protective plastic wrapper that fully encloses the article. The protective wrapper is permanently affixed to the roll axially in a 270° arc, the remaining 90° arc forming an axially extending flap tethered to the roll. In some embodiments, the absorbent article may be distributed in a kit with a lubricant. The lubricant is a petroleum-free, water-based moisturizing balm used on the absorbent material to moisturize vaginal tissues and prevent dryness. In other embodiments, the absorbent material is pre-moisturized with a lubricant.

### BACKGROUND OF THE INVENTION

1. Field of the Invention

The present invention relates to sanitary products, and particularly to an absorbent article for incontinent women that helps to prevent clothing stains from urinary incontinence, and that may also reduce or eliminate problems with urinary incontinence through retraining or reconditioning the muscles for better control of micturition.

2. Description of the Related Art

Incontinence is a medical problem that can hamper one's quality of life. Female urinary incontinence (UI) may be treated using both intrusive and less intrusive measures. In extreme cases, UI may be treated with surgery, hormones or drugs. Because of their invasive nature, these treatment methods are generally used as last resorts. In less severe cases of UI, individuals may be advised to strengthen their pelvic floor muscles by doing Kegel exercises or other pelvic muscle exercises. These exercises are effective if properly performed o a regular basis. Many individuals may lack sufficient discipline to perform the exercises regularly, or improperly perform the exercises, or find the exercises difficult to perform and/or uncomfortable to carry out.

Still other devices used by incontinent women are simply garments designed for controlling the embarrassing effects of lack of urinary control. These incontinence devices include drainage bags or incontinence garments. Although these devices allow individuals to regain a sense of control and dignity, and to live life in a somewhat normal manner, the degree of freedom that can be regained is restricted. Drainage bags are often used with catheters or other such devices, while diapers are fitted around the entire pelvic region of the user. Both devices require clothing that can be worn loosely.

Thus, a device is desired that will allow a user to protect clothing from accidental urine leakage. The device should be easily worn without encumbering other areas of the body or restricting the type of clothing that must be worn to accommodate the device. It would be further desirable that such a device helps to relieve incontinence through isometric or other muscular retraining or reconditioning through use of the device. Thus, an absorbent article for incontinent women solving the aforementioned problems is desired.

### SUMMARY OF THE INVENTION

The absorbent article for incontinent women is a cylinder of absorbent material that is disposed between the labia majora, just under or in front of the urethra. The article is designed to absorb urine accidentally discharged by the body. The article may be made solely as a cylinder of absorbent material, or as a cylinder of absorbent material disposed in a protective plastic wrapper. The wrapper encloses the article axially. The wrapper includes an axially extending flap defined by parallel perforations in the wrapper. The flap is attached to the cylinder of absorbent material by adhesive and tethered to one end of the cylinder.

In use, the user lifts the flap by pulling the tab to tear the perforations and folds the flap back to expose cylinder of absorbent material. The exposed portion of the absorbent article is then positioned to face the urethra. The portion of the absorbent article that is still enclosed in the wrapper is positioned to face the user's undergarment. By placing the absorbent article in this manner, the wrapper protects the undergarments from fluids leaking from the body. With the absorbent article placed between the labia, the flap is positioned over the mons pubis, away from the vulva. The flap provides a handle to remove the absorbent article without soiling one's hand.

In some embodiments, the absorbent article may be distributed in a kit. The kit provides a lubricant for use with the absorbent article. The lubricant is applied directly to the absorbent article to moisturize vaginal tissues and prevent dryness. The lubricant can be a petroleum-free, water-based moisturizing balm.

These and other features of the present invention will become readily apparent upon further review of the following specification and drawings.

### BRIEF DESCRIPTION OF THE DRAWINGS

FIG. 1 is a perspective view of a kit having an absorbent article for incontinent women and a lubricant according to the present invention.

FIG. 2 is a perspective view of an alternative embodiment of an absorbent article for incontinent women disposed in a protective wrapper.

FIG. 3 is a perspective view of the absorbent article of FIG. 2 with the flap of the protective wrapper pulled back from the absorbent article.

FIG. 4 is a diagrammatic, environmental side view of the lower portion of a female with the absorbent article for incontinent women of the present invention shown disposed between the labia majora.

FIG. 5 is a diagrammatic section view drawn along lines 5-5 of FIG. 4.

Similar reference characters denote corresponding features consistently throughout the attached drawings.

### DETAILED DESCRIPTION OF THE PREFERRED EMBODIMENTS

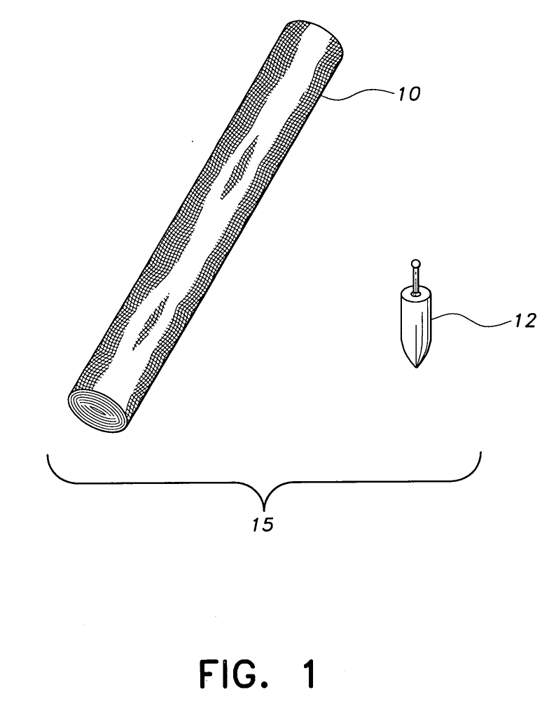

The present invention is an absorbent article for incontinent women, designated as 10 in the figures. As shown in FIG. 1, the absorbent article 10 is a roll or cylinder of absorbent material. The absorbent article 10 is made of an absorbent non-irritating, non-toxic substance, such as cotton fibers, that easily absorbs fluids. In some embodiments, the absorbent article 10 may be provided as a kit 15 that contains the article 10 in combination with a lubricant 12.

The absorbent article 10 has a length and a diameter dimensioned and configured to fit between the labia majora, extending into the vulva horizontally (i.e., from front to back, or ventral to dorsal) to apply pressure to the urethra. The absorbent article 10 may be made in different sizes: small, medium and large. The small size absorbent article 10 is about three and one-quarter inches long and one-half inch in diameter. The medium size absorbent article 10 is about three and one-half inches long and three-quarters of an inch in diameter. The large size absorbent article 10 is about four inches long and three-quarter of an inch in diameter. The above dimensions are representative only, and are not to be considered limiting; for example, an absorbent article two inches long and one-half inch in diameter may be used to advantage.

The lubricant 12 is a petroleum-free, water-based moisturizing balm designed for use directly on the absorbent article 10. While in some cases the absorbent article may be used dry, the lubricant 12 helps to ensure that the vaginal tissues are moisturized to avoid discomfort, and that the absorbent article 10 can be easily slid in place between the folds of the labia majora. The lubricant 12 may be provided in an easy to use single-use capsule that can be carried discreetly and conveniently in a purse or pocket.

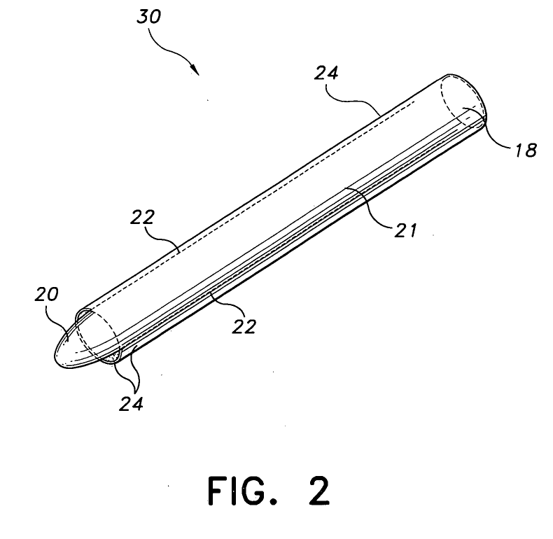

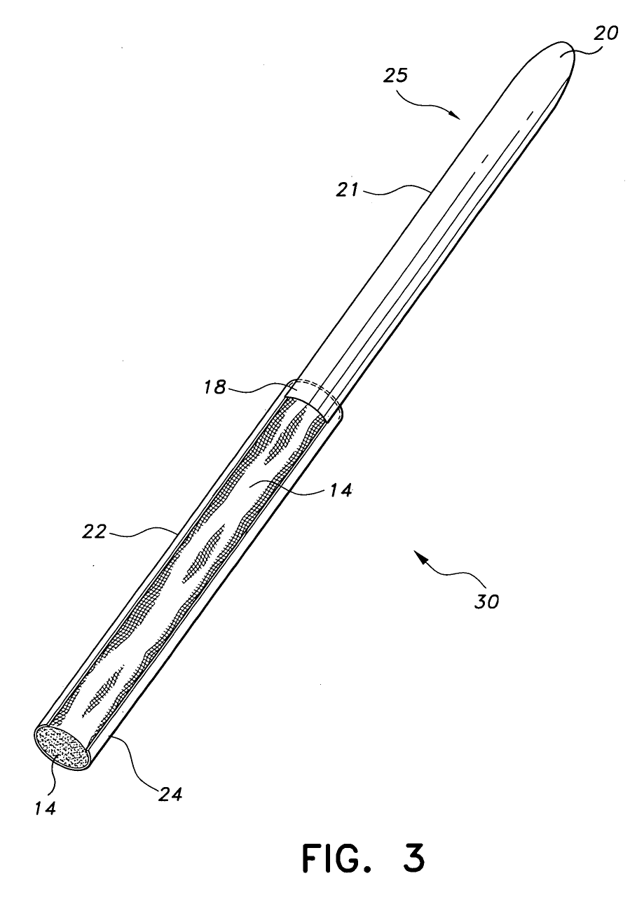

Referring now to FIGS. 2 and 3, an embodiment of an absorbent article designated as a sheathed absorbent article 30 shows the basic absorbent article, or cylindrical roll 14 of absorbent material, disposed in a protective plastic wrapper 24. The sheathed absorbent article 30 is packaged with the wrapper 24 fully enclosing at least the sides of the cylindrical roll 14, although the ends may also be enclosed, if desired. The wrapper 24 is permanently affixed to the roll of absorbent material in an arc of about 270° about the circumference of the cylindrical roll 14 throughout its axial length. Along the remaining 90° arc, the wrapper 24 has an end 18 fixed to the roll 14, a tab 20 that extends from the opposite end, and an elongated central section 21 attached to the roll 14 by a non-allergenic adhesive, with parallel elongate rows of perforations 22 extending between the tab 20 and the fixed end 18. When ready for use, the tab 20 is pulled back, separating the perforations 22, so that the tab 20 and the central section 21 form a flap 25 folded back over the fixed end 18 and the cylindrical roll 14 is exposed. When the sheathed absorbent article 30 is lodged in the vulva, the flap 25 extends external to the vulva and is used to remove the absorbent article 30 without soiling the hands. The cylindrical roll 14 may be pre-lubricated, if desired.

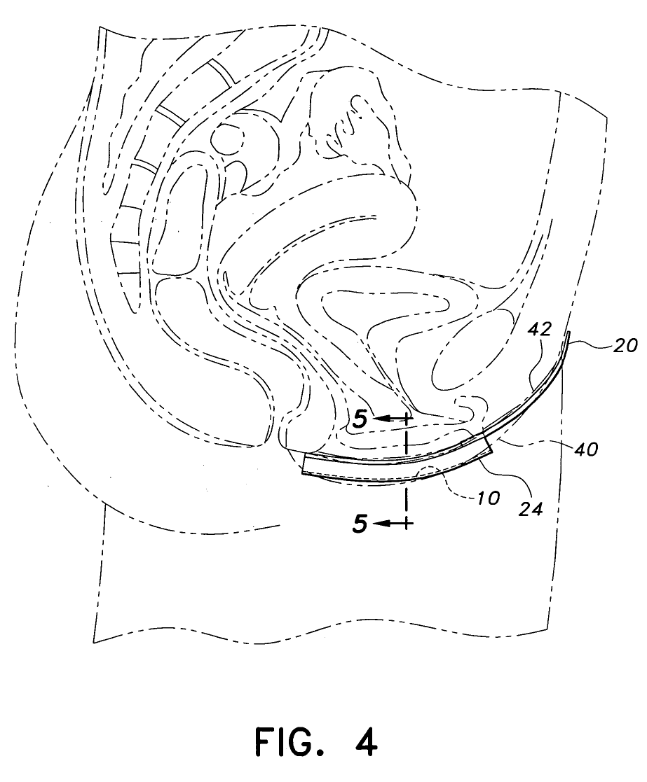

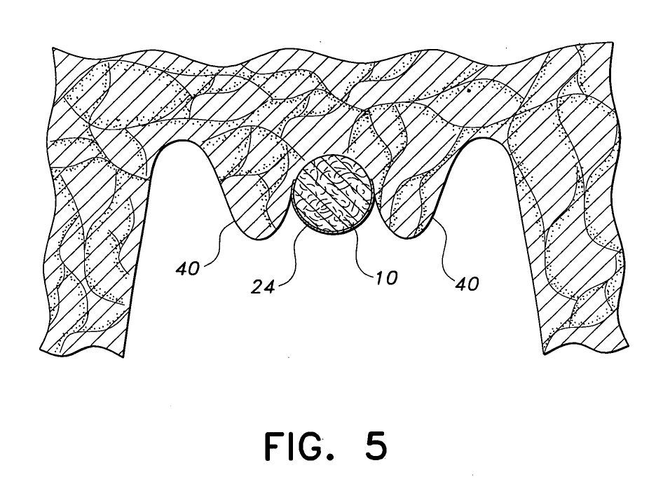

Referring now to FIGS. 4 and 5, a user will prepare the sheathed absorbent article 30 for use by exposing a portion of the cylindrical roll 14. To do this, the user will lift tab 20 and pull back the perforated central portion 21, and leaving fixed end 18 attached to the cylindrical roll 14. As with absorbent article 10, the user may apply lubricant 12 to the cylindrical roll 14, if desired, or roll 14 may be pre-lubricated with lubricant and vaginal moisturizers.

The absorbent article 30 is oriented in the vulva by positioning the exposed portion of the cylindrical roll 14 to face the urethra. The wrapper 24 faces the user's undergarment 40. By placing the absorbent article 30 in this manner, the wrapper 24 serves as a trough to retain excess liquids that may leak if the cylindrical roll 14 has become saturated. The wrapper 24 protects the undergarments from fluid leaks.

Unlike a tampon, which is usually placed in the vagina, the sheathed absorbent article 30, like the absorbent article 10, is placed in the vulva or external genitalia, being lodged in the folds of the labia majora 40. The flap 25 is placed over the mons pubis 42, away from the vulva. The flap 25 provides a handle to remove the absorbent article 30 with without soiling one's hands.

It is believed that when using either the absorbent article 10 or the absorbent article 30, the user will unconsciously compress the pelvic floor muscles to retain the absorbent article 10 or 30 between labia majora 40, and so that pressure may be placed against the urethra.

As a result, the user will isometrically strengthen the pelvic floor muscles and may eventually be able to regain control over micturition, thereby relieving urinary incontinence.

It is to be understood that the present invention is not limited to the embodiments described above, but encompasses any and all embodiments within the scope of the following claims.

1-17. (canceled)

18. An absorbent article for protecting a woman's clothing and relieving female urinary incontinence, said absorbent article comprising:
a cylindrical roll of absorbent material having opposite ends and an elongate circumferential surface extending between the opposite ends, said cylindrical roll further having a diameter dimensioned and configured to fit between a woman's labia majora; and
a protective wrapper disposed at least about the circumferential surface of said cylindrical roll, said protective wrapper including an arcuate section adhesively affixed to a major portion of the circumferential surface of said cylindrical roll and a separable elongated section having a first end and an opposite second end, the separable section being bounded by parallel rows of perforations extending between the opposite ends of said cylindrical roll, wherein a tab extends from the first end of the separable section and the second end is affixed to said cylindrical roll;
whereby, upon pulling the tab by a user to separate the rows of perforations, the separable section forms a flap to expose a minor portion of the cylindrical roll;
whereby, the user then orients the absorbent article in the user's vulva between the user's labia majora by positioning the exposed portion of the cylindrical roll to face the user's urethra with the protective wrapper facing the user's undergarment; and
whereby, continued use of the absorbent article isometrically strengthens the user's pelvic floor muscles, thereby relieving urinary incontinence.

19. The absorbent article according to claim 18, wherein said protective wrapper is made from plastic.

20. The absorbent article according to claim 18, wherein the arcuate section of said protective wrapper forms a 270° arc about said cylindrical roll.

21. The absorbent article according to claim 18, wherein the exposed portion of said cylindrical roll is pre-lubricated with a vaginal lubricant.

22. The absorbent article according to claim 21, wherein the lubricant is a moisturizing balm.
